(19) United States
(12) Patent Application Publication
(10) Pub. No.: US 2025/0019659 A1
(43) Pub. Date: Jan. 16, 2025

(54) Method for Obtaining Cultures of Mesenchymal Stem Cells (MSCs) from the Perivascular Space of the Umbilical Cord Vessel

(71) Applicant: BAOSHENGTANG BIOTECH CO., LTD., Taichung City (TW)

(72) Inventor: Ding-Yin Huang, Taichung City (TW)

(21) Appl. No.: 18/350,892

(22) Filed: Jul. 12, 2023

(51) Int. Cl. C12N 5/0775

(52) U.S. Cl. CPC C12N 5/0662; C12N 2501/734; C12N 2506/025

(57) ABSTRACT

The method of the present invention includes cutting out a umbilical cord segment; filling the vessel of the umbilical cord segment with 0.01-0.5% collagenase solution; sealing both ends of the umbilical cord segment and maintaining it at a temperature of 37° C. for at least 15-45 minutes; wash the vessel with balanced normal saline solution; fill the vessel with a new 0.01-0.5% collagenase solution; seal both ends of the umbilical cord and incubate at 37° C. for at least 25-65 minutes; and equilibrate The vessel is washed with saline solution, the washed liquid is centrifuged, and the resulting pellet is incubated in a selective environment. The present invention allows to increase the yield and purity of obtained mesenchymal stem cell cultures and in some cases can reduce the time required to obtain mesenchymal stem cells.

### FIELD OF THE INVENTION

The present invention is related to the field of cell biology and biotechnology, particularly related to cell-related technology, especially related to the preparation method of mesenchymal stem cells for regenerative treatment.

### BACKGROUND OF THE INVENTION

Mesenchymal stromal (stem) cells (MSCs) are multifunctional cells derived from mesoderm that are capable of self-sustaining populations and differentiating into mesenchymal cells such as chondrocytes, bone cells, and adipocytes. There are currently several methods to differentiate MSCs into cells such as epithelial cells, neuron-like cells, etc. formed from other germ layers in vivo. MSCs have immunomodulatory effects, can secrete angiogenic factors, and can migrate to injury sites. In addition, MSCs can also differentiate into cells of damaged tissues, thereby MSCs are helpful in restoring the functional integrity of organs or tissues. These properties make MSCs a promising tool for cell technology-based regenerative therapies.

The umbilical cord (particularly the Wharton's gel in it and the perivascular space), as well as cord blood and placenta, which are usually discarded after delivery, are convenient sources of MSCs because they contain a large number of MSCs in 1 gram of tissue as compared with other tissue sources, therefore the total number of MSCs that can be obtained is also higher. In addition, as compared with adult MSCs obtained from adipose tissue and bone marrow, cells from these tissue sources also have a higher proliferative potential, which allows to reduce the expansion time, that is, cells can survive in vitro without loss of differentiation potential. In most cases, such expansion process is necessary for most therapeutic and surgical procedures using MSCs, because no source of MSCs can obtain sufficient cell numbers for adult patients without expansion, that is, approximately 20-80 million cells depending on body weight and treatment steps on a patient-by-patient basis.

Five regions in the umbilical cord from which MSCs can be isolated have been identified:
1. 20-50% of freshly isolated mononuclear cell fraction from umbilical cord blood;
2. umbilical vein subendothelial layer;
3. the outer layer of the umbilical cord vessels (perivascular space);
4. intravascular space;
5. subamniotic space.

MSCs isolated from different regions of the umbilical cord are different populations with the same immunophenotype, but different proliferative activity and differentiation ability, and these data are reviewed and described in the following article: Conconi et al, Phenotype and Differentiation Potential of Stromal Populations Obtained from various Zones of Human Umbilical Cord: An Overview. The Open Tissue Engineering and Regenerative Medicine Journal. 2011; 4: 6-20. MSCs present in the perivascular space are characterized by their high in vitro population growth rate, as well as high expression levels of pancytokeratin antigen and CD146 [Carvalho M., Teixeira F G, Reis R L, Sousa N., Salgado A J. Mesenchymal stem cells in the umbilical cord: phenotypic characterization, secretome and applications in central nervous system regenerative medicine. Curr Stem Cell Res Ther. 2011 September; 6(3):221-8]. In addition, MSCs from these regions also have differences in the production levels of IL-6 and FLT3 [Xu M., Zhang B., Liu Y., Zhang J., Sheng H., Shi R., Chen H. The immunological and hematopoietic profiles of mesenchymal stem cells derived from different sections of human umbilical cord. Acta Biochimica et Biophysica Sinica, Volume 46, Issue 12, December 2014, Pages 1056-1065].

In the following articles: Ennis J., Sarugaser R., Gomez A., Baksh D., Davies J E Isolation, characterization, and differentiation of human umbilical cord perivascular cells (HUCPVCs). Methods Cell Biol. 2008; 86: 121-36, presented the feasibility of obtaining MSCs from the perivascular space of the umbilical vein.

Currently known methods for obtaining MSC cultures from the perivascular space of the umbilical cord are described in: Sarugaser R., Lickorish D., Baksh D., Hosseini M M, Davies J E Human umbilical cord perivascular (HUCPV) cells: a source of mesenchymal progenitors. Stem Cells. 2005 February; 23(2): 220-9. This method is carried out by cutting the blood vessels of the umbilical cord—the vein and the two arteries—individually from Wharton's gel, suturing the ends of each vessel together to form a closed loop, and culturing in 0.1% collagenase solution for 18-24 hours. The loops are then removed and the solution are centrifuged to obtain precipitation pellet. The pelleted are washed for several times and incubated in a standard manner. The above-mentioned method including the complicated procedure of preparation from Wharton's gel and the enzymatic treatment process of up to 24 hours and is very time-consuming, which makes the above-mentioned method not suitable for mass therapeutic use of large number of patients, especially when it is necessary to obtain MSCs quickly. At the same time, the viability of the isolated cells obtain from this method was also very low due to the long-term enzyme treatment (24 hours).

In the following articles: Aizenshtadt A A, Ivanova N A, Bagaeva V V, Smolyaninov A B, Samoilovich M P, Klimovich V B Intracellular immunoglobulins in the Namalva and U266 lines upon co-cultivation with mesenchymal cells. Tsitologiia 2014; 56(2): 2, 117-121, described a method to obtain MSCs from the perivascular space of umbilical cords, which can reduce the processing time of umbilical cords from 16-26 hours to 1.5 hours. This method is characterized in that the umbilical vein is a complete unground umbilical cord fragment (10-30 cm), and 0.2% type IV collagenase solution is introduced to the umbilical cord fragment followed by sealing the both ends of the umbilical cord fragment and then incubating at 37° C. for 60 minutes. After incubation, the umbilical veins were washed with phosphate buffered saline, and the obtained liquid were centrifuged. the precipitation pellet was transferred to selective medium and subculture for several passages in culture vessels suitable for culturing adhesive cells.

The obtained cells have the immunophenotype of MSCs and can differentiate towards bone, cartilage and adipocytes. The above method significantly reduces the time to obtain MSCs culture by removing the preparation stage of umbilical cord vessels and reducing the time of collagenase treatment. However, the disadvantages of this method includes a) low cell yield—about 1 million cells per 10 cm of umbilical cord length after the first subculture; MSCs pollution; b) contamination of MSCs with other types of cells, mainly endotheliocytes; c) the safety of the obtained MSCs is low due to long-term culture in collagenase solution for 1 hour, and therefore their proliferative activity is also quite low; d) the expansion and culture time of MSCs is longer (5 days). These disadvantages make the above methods not suitable for commercial use.

### SUMMARY OF THE INVENTION

The object of the present invention is to provide a method for producing MSCs culture from the perivascular space of the umbilical cord vessels, which can reduce the time required to obtain MSCs culture, improve the yield and safety of the obtained cells, and improves the purity of the culture of MSCs.

The above problems in the prior art are solved by a method of obtaining MSCs culture from the perivascular space of the umbilical cord, wherein the method comprises cutting out a section of umbilical cord; filling the umbilical cord vessel with 0.01-0.5% collagenase solution; sealing both ends of the umbilical cord, and incubating the umbilical cord at 37° C. for 15-45 minutes; then washing the vessel with a first balanced normal saline solution and harvesting the first balanced salt solution, filling the vessel with a new 0.01-0.5% collagenase solution, sealing the two ends of the umbilical cord again, and maintain the umbilical cord at 37° C. for 25-65 minutes, washing the vessel with a second balanced salt solution and harvesting the second balanced salt solution. The first and second balanced salt solution are centrifugated to obtained precipitating pellets which are then cultivated with a known manner using a selective medium.

The difference between the present invention and the prior art resides in that the method of the present invention for producing MSCs culture carried out two incubating cycles in collagenase solution. When the umbilical cord was incubated in the umbilical vessel cavity with 0.2% collagenase solution for merely the first time, only part of the extracellular matrix of the umbilical vessel wall was destroyed. Therefore, the solution collected from the umbilical vessel at this time mainly contained cells from zone 1-2 of the umbilical cord. That is to say, besides MSCs, there are a large number of endothelial cells, blood cells, hematopoietic stem cells and so on in this fraction. Whereas after the second incubation, the obtained fraction mainly contains cells of zone 3-4, which is usually absent of Wharton's gel mucopolysaccharide (zone 5). With subsequent washing procedure, excess collagenase will be removed, therefore most harvested cells will not be damaged by the collagenase. By implementing the present method, it is possible to increase the proportion of MSCs in the preparation of isolated cells, reduce the overall time for obtaining cell cultures, increase cell safety and cell culture purity, increase the proportion of actively proliferating perivascular space MSCs, and reduce the percentage or even completely absent of slow-growing Wharton's gel MSCs. Thereby, the MSCs culture obtained by implementing the method of the present invention can maintain the viability and proliferation potential thereof.

If necessary, the two cell fractions obtained after the first and second incubation with collagen were cultured on selective media using adhesive plastic discs, and the resulting MSCs cultures were combined for use in regenerative therapy.

In a specific embodiment, the concentration of the collagenase solution is preferably 0.1-0.3%, most preferably 0.2%.

In another specific embodiment, the first incubation is preferably carried out for 25-45 minutes, more preferably for 25-35 minutes, most preferably for 30 minutes.

In yet another specific embodiment, the second incubation is preferably carried out for 40-65 minutes, more preferably for 40-50 minutes, most preferably for 45 minutes.

The steps and sequence of operations procedure of the method of the present invention for processing the obtained umbilical cord segments and the instruments and materials for selection as described above are used to provide a solution of known problems in the prior art.

### BRIEF DESCRIPTION OF THE DRAWINGS

The above contents of the present disclosure will become more readily apparent to those ordinarily skilled in the art after reviewing the following detailed description and accompanying drawings, in which:

FIG. 1 shows the number of MSCs and other types of cells obtained after the first subculture, which depend on the exposure time to 0.2% collagenase solution of the first and second incubation.

FIG. 1 shows the number of MSCs and other types of cells obtained by the method described in Example 1, as identified on the figure.

FIG. 1 shows the number of MSCs and other types of cells obtained by the method described in Example 2, as identified on the figure.

FIG. 1 shows the number of MSCs and other types of cells obtained by the method described in Example 3, as identified on the figure.

FIGS. 2A-1, 2A-2, 2B-1, 2B-2, 2C-1, and 2C-2 show the first and second exposures to Microscopic images of cell cultures obtained in 0.2% collagenase solution.

FIG. 2A-1 shows a microscopic image of cell cultures obtained as described in Example 1 after exposure to 0.2% collagenase solution for 20 minutes in the first incubation.

FIG. 2A-2 shows a microscopic image of cell cultures obtained as described in Example 1 after exposure to 0.2% collagenase solution for 30 minutes in the second incubation.

FIG. 2B-1 shows a microscopic image of cell cultures obtained as described in Example 2 after exposure to 0.2% collagenase solution for 30 minutes in the first incubation.

FIG. 2B-2 shows a microscopic image of cell cultures obtained as described in Example 2 after exposure to 0.2% collagenase solution for 45 minutes in the first incubation.

FIG. 2C-1 shows a microscopic image of cell cultures obtained as described in Example 3 after exposure to 0.2% collagenase solution for 40 minutes in the first incubation.

FIG. 2C-2 shows a microscopic image of cell cultures obtained as described in Example 3 after exposure to 0.2% collagenase solution for 60 minutes in the first incubation.

These images were acquired by using a laboratory research biological microscope (Axiovert 40 series with Axiovert 40 CFL tripod) and a microscope camera (Progress ST 3 Carl Zeiss, Germany).

### DETAILED DESCRIPTION OF THE PREFERRED EMBODIMENT

The method of the present invention is carried out as follows.

### Example 1

Umbilical cords obtained in a standard manner were placed in containers containing a sufficient amount of “heparin water” (5 ml heparin/200 ml normal saline) and antibiotics added, and then transported to the laboratory. Thoroughly flush the umbilical vessel with a balanced saline solution (e.g., Versen's solution) using a syringe. The umbilical vessel was then washed with a 0.2% solution of type I and type IV collagenase (1:1) in PBS using a syringe. One side of the umbilical cord was sealed, and then the umbilical vessel was filled with the collagenase solution described above. The opposite end of the umbilical cord is clamped. The sample is placed in a Petri dish and kept in a shaking incubator, such as ES-20 (Biosan, Latvia), at 37° C. with gentle agitation for 20 minutes. After this, the collagenase solution was collected and the umbilical vessel was washed thoroughly with a balanced saline solution (e.g., PBS solution). The washed collagenase solution and saline solution are collected and centrifuged at 1100 rpm for 7 min in a centrifuge, e.g., an SM-6M centrifuge (Elmi, Latvia). Remove the supernatant, and place the pellet in a 75 cm² culture flasks pre-added with 10-15 ml of complete medium. The complete medium is, for example, MEM-α/Advanced Stem Cell Media (HyClone, Germany) supplemented with 20% fetal bovine serum for MSCs, or Serum Replacement Advanced Supplement (HyClone, Germany) for stem cells Germany). The flask is placed in an incubator provided with CO2 atmosphere, such as BBD 6220 (Thermo, Germany), to allow MSCs to partially attach to the plastic; the exposure time is 3-5 days, depending on the state of the cells. The first fraction of MSCs obtained was used for comparison. The umbilical vessel was washed for a second time with the collagenase solution using a syringe. One side of the umbilical cord was sealed, and then the umbilical vessel was filled with the collagenase solution described above. The opposite end of the umbilical cord is clamped. The sample was placed in a Petri dish and stirred gently for 30 minutes. After standing, the collagenase solution was collected and the umbilical vessel was thoroughly washed with the balanced physiological saline solution. The washed collagenase solution and saline solution are collected and centrifuged at 1100 rpm for 7 min in an SM-6M centrifuge (Elmi, Latvia). The supernatant was removed and the pellet was placed in a 75 cm² culture flask added with 10-15 ml of complete medium previously. The culture flask is placed in the CO2 incubator as described above to allow the MSCs to partially attach to the plastic; the exposure time is 3-5 days, depending on the state of the cells. Next, the MSCs are cultured by a known MSCs culture method.

### Example 2

The preparation of MSCs culture is carried out according to the procedure shown in embodiment 1 characterized in that the first incubation is exposed to the collagenase solution for 30 minutes and the second incubation is exposed to the collagenase solution for 45 minutes.

### Example 3

The MSCs culture obtained according to the procedure shown in embodiment 1 is characterized in that the first incubation is exposed to the collagenase solution for 40 minutes and the second incubation is exposed to the collagenase solution for 60 minutes.

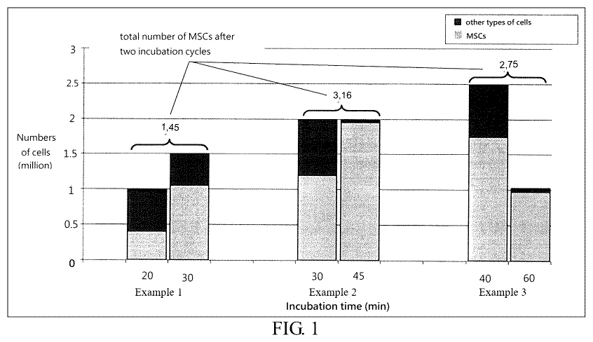

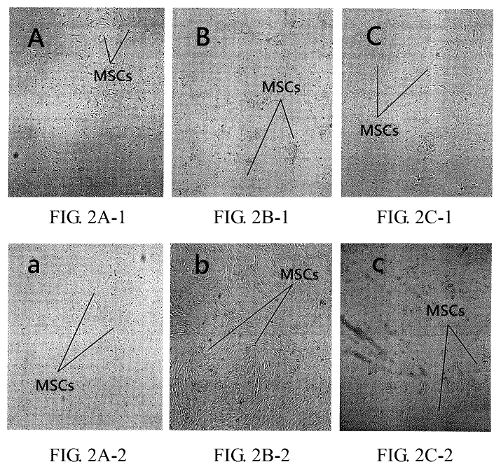

As shown in FIG. 1, the proportion of MSCs obtained from the second incubation is higher under the operation conditions in 1b (first incubation for 30 minutes and second incubation for 45 minutes) and 1c (first incubation for 40 minutes and second incubation for 60 minutes). Furthermore, the total number of MSCs obtained from the first and the second incubation is higher under the operation conditions in 1b (first incubation for 30 minutes and second incubation for 45 minutes).

What is claimed is:

1. A method for obtaining mesenchymal stem cell culture from the perivascular space around the umbilical cord blood vessel, wherein the method comprises
cutting out a section of umbilical cord;
filling the umbilical cord vessel with 0.01-0.5% collagenase solution;
sealing both ends of the umbilical cord and incubating the umbilical cord at 37° C. for 15-45 minutes for first incubation;
washing the vessel with a first balanced normal saline;
filling the vessel with a new 0.01-0.5% collagenase solution;
sealing the two ends of the umbilical cord again and incubating the umbilical cord at 37° C. for 25-65 minutes for second incubation;
washing the vessel with a second balanced salt solution and harvesting the second balanced salt solution; and
centrifuging the harvested second balanced salt solution and culturing the pellet obtained from centrifugation on a selective medium.

2. The method according to claim 1, wherein the concentration of the collagenase solution is 0.1-0.3%.

3. The method according to claim 1, wherein the concentration of the collagenase solution is 0.2%.

4. The method according to claim 1, wherein the first incubation is carried out for 25-45 minutes.

5. The method according to claim 1, wherein the first incubation is carried out for 25-35 minutes.

6. The method according to claim 1, wherein the first incubation is carried out for 30 minutes.

7. The method according to claim 1, wherein the second incubation is carried out for 40-65 minutes.

8. The method according to claim 1, wherein the second incubation is carried out for 40-50 minutes.

9. The method according to claim 1, wherein the second incubation is carried out for 45 minutes.

10. A method for obtaining mesenchymal stem cell culture from the perivascular space around the umbilical cord blood vessel, wherein the method comprises
cutting out a section of umbilical cord;
filling the umbilical cord vessel with 0.01-0.5% collagenase solution;
sealing both ends of the umbilical cord and incubating the umbilical cord at 37° C. for 15-45 minutes for first incubation;
washing the vessel with a first balanced normal saline solution and harvesting the first balanced salt solution;
centrifuging the first balanced salt solution;
filling the vessel with a new 0.01-0.5% collagenase solution;
sealing the two ends of the umbilical cord again and incubating the umbilical cord at 37° C. for 25-65 minutes for second incubation;
washing the vessel with a second balanced salt solution and harvesting the second balanced salt solution; and
centrifuging the harvested first and second balanced salt solution and culturing the pellet obtained from centrifugation on a selective medium.

11. The method according to claim 10, wherein the concentration of the collagenase solution is 0.1-0.3%.

12. The method according to claim 10, wherein the concentration of the collagenase solution is 0.2%.

13. The method according to claim 10, wherein the first incubation is carried out for 25-45 minutes.

14. The method according to claim item 10, wherein the first incubation is carried out for 25-35 minutes.

15. The method according to claim 10, wherein the first incubation is carried out for 30 minutes.

16. The method of according to claim 10, wherein the second incubation is carried out for 40-65 minutes.

17. The method according to claim 10, wherein the second incubation is carried out for 40-50 minutes.

18. The method of according to claim 10, wherein the second incubation is carried out for 45 minutes.
